(19) United States
(12) Patent Application Publication
(10) Pub. No.: US 2017/0056125 A1
(43) Pub. Date: Mar. 2, 2017

(54) MEDICAL ASSESSMENT KIT AND METHOD

(71) Applicants: Christopher R. Garza, Wichita Falls, TX (US); Dalila G. Garza, Wichita Falls, TX (US); Ida A. Gomez, Wichita Falls, TX (US)

(72) Inventors: Christopher R. Garza, Wichita Falls, TX (US); Dalila G. Garza, Wichita Falls, TX (US); Ida A. Gomez, Wichita Falls, TX (US)

(21) Appl. No.: 14/833,244

(22) Filed: Aug. 24, 2015

(51) Int. Cl. A61B 90/40; A61B 50/31; A61B 42/10; A61B 5/00; A61B 42/40; A61B 50/36; A61B 46/10; A61B 90/70

(52) U.S. Cl. CPC A61B 90/40; A61B 46/10; A61B 50/312; A61B 90/70; A61B 5/00; A61B 42/40; A61B 50/36; A61B 42/10; A61B 2050/311; A61B 2505/07; A61B 2050/3008

(57) ABSTRACT

The present application includes a medical assessment kit for ensuring compliance with regulations during the assessment of a patient. The kit is carried by a carrying device and is configured to hold items to provide a sanitary work space free of cross contamination. Various assessment tools are used in combination with the kit and are transported between a clean area and a dirty area as denoted by various liners. Protection devices may be used by an operator to ensure protection to the medical professional. The kit allows the medical professional to sanitarily perform the assessment and dispose of necessary items in accordance with local regulations.

### BACKGROUND

1. Field of the Invention

The present application relates generally to medical assessment kits and, more particularly, to a kit for use in providing home medical assessments in compliance with applicable regulations.

2. Description of Related Art

A large number of individuals are suffering from any number of ailments that necessitate relatively frequent health supervision. These individuals may be home bound or limited in their physical capacities. In such situations, home health medical professional are often used to track and monitor the health of these individuals.

During visits and while performing treatment and assessments, the home health professional uses a collection of tools, techniques, and items to accurately assess any number of physical characteristics, such as blood pressure, temperature, breathing, sores, and so forth. It is imperative that proper sanitization and cleanliness be maintained in compliance with state and national regulations. Regulations require the need to maintain a sanitary workspace and to prevent the cross contamination of tools and/or items during and after use. Although being monitored to some level and encouraged, such standards are often ignored or unattained. Failure to maintain regulations and compliance standards can result in fines. A contributing factor to the low compliance is that there is generally a lack of viable kits standard in the industry for home health medical professionals to use.

It is desirable to provide a medical assessment kit that provides the tools and items necessary to home health medical professionals to ensure compliance with applicable regulations. Although great strides have been made, considerable shortcomings remain.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a chart of a medical assessment kit according to the preferred embodiment of the present application;

FIG. 2 is a top view of the medical assessment kit of FIG. 1 partially laid open; and

FIG. 3 is a chart of the method of using the medical assessment kit of FIG. 1.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system in accordance with the present application overcomes one or more of the above-discussed problems commonly associated with conventional medical assessment practices and procedures. Specifically, the system of the present application is configured to provide an environment free of cross contamination for the patient and medical professional during a home health assessment. This and other unique features of the system are discussed below and illustrated in the accompanying drawings.

The system and method will be understood, both as to its substance and operation, from the accompanying drawings, taken in conjunction with the accompanying description. Several embodiments of the system are contemplated herein. It should be understood that various components, items, and features of the system and method may be modified and/or interchanged with one another, all of which are within the scope of the present application, even though not all variations and particular embodiments are shown in the drawings. It should also be understood that the mixing and matching of features, elements, and/or functions to form various embodiments is expressly contemplated herein so that one of ordinary skill in the art would appreciate from this disclosure that the features, elements, and/or functions of one embodiment may be modified into another embodiment as appropriate, unless otherwise described.

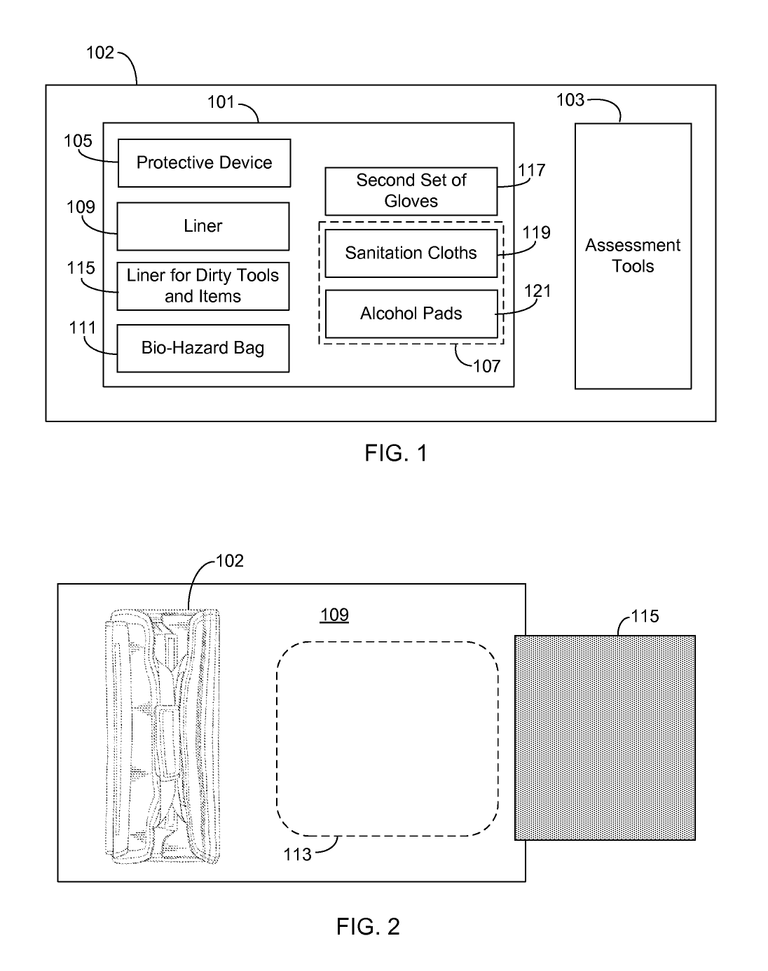

The medical assessment kit/system and method of the present application is illustrated in the associated drawings. Referring now to the drawings wherein like reference characters identify corresponding or similar elements in form and function throughout the several views. FIGS. 1-2 in the drawings illustrate a medical assessment kit 101. Kit 101 is configured to contain the tools and items necessary to permit a medical professional the ability to perform a home health assessment of a patient in compliance with regulations. Kit 101 is configured to be carried within a single carrying device 102.

Kit 101 is transported within carrying device 102 which is configured to hold kit 101 and other items (i.e. tools 103) to provide a sanitary work space free of cross contamination during the assessment. Kit 101 may include may include at least the following items: 1) a liner 109 and 115; 2) a protection device 105 and 117; 3) and a cleaning device 107. Assessment tools 103 are also carried within device 102 and the exact items within it are dependent upon the type of assessment performed. Other items may be included in kit 101, such as a bio-hazard bag 111. Each of the items/tools are brought out of carrying device 102 at an appropriate time for a particular purpose. It is a goal of the present application that kit 101 be used and configured to provide separate areas for clean and dirty items and tools. Also that items may be transferred between each clean and dirty area as further described below.

Carrying device 102 is illustrated from a top view more completely in FIG. 2. In this figure, device 102 is a bag having a plurality of pockets both internally and externally for storage of the various items or tools, and straps to aid in mobility from location to location. This particular depiction of carrying device 102 is not meant to be limiting. Device 102 may be any of a bag, a case (i.e. suitcase or hardened and formed bag), or a paper based rigid structure (i.e. a box or chest). Although carrying device 102 has been described as a single device to carry all tools and items necessary for performing the assessment, it is understood that a plurality of carrying devices 102 may be used in kit 101.

Assessment tools 103 are any of a plurality of types of common tools used by medical professionals to assess a number of conditions of a patient. With tools 103, the medical professional may assess blood pressure, temperature, skin conditions, breathing, and so forth. Common tools may be used to perform these and other assessments. Tools 103 may be either a one-time use tool or be reusable for multiple assessments. If only good for a one-time use, the tool will be disposed of after the assessment. Reusable tools are cleaned and reinserted into carrying device 102 for the next assessment.

A key feature to kit 101 is the ability to separate device 102 and all the items from contact with the property of the patient. It is imperative that the tools and items stay sanitary prior to use and are either disposed of or cleaned after use. This requires the medical professional to establish a barrier between device 102, kit 101, and tools 103 from any object(s) they rest on. Liner 109 is configured to be laid beneath device 102 and establish a working area identified as a clean area 113. Clean area 113 is for the resting of tools and items as they are removed from device 102 but prior to use. A second liner 115 is laid adjacent to liner 109 and also acts as a barrier to establish a working area identified as a dirty area. As tools and items within kit 101 are used, they are placed onto liner 115. Second liner 115 may be located adjacent to liner 113 for designating a dirty area for the placing of used items and tools that are now considered contaminated. However, liner 115 does not necessarily need to overlap liner 109, nor does liner 115 need to be immediately adjacent. Liner 115 may be remotely located from liner 109.

Protection device 105 acts as a barrier between the medical professional and both the patient and any biological substances. Its purpose is to ensure the cleanliness and protection of the medical professional. Such devices 105 may be worn by the medical professional, such as a pair of gloves, mask, apron, and so forth. Other devices 105 may be erectable devices to shield the medical professional, such as screens or shields. It is understood that a plurality of devices 105 may be used. Additionally a second pair of gloves 117 may be available in kit 101 to allow the medical professional to exchange or replace as necessary.

Cleaning devices 107 are used to sterilize reusable tools and items after use and provide proper sanitation to the medical professional. Examples of cleaning devices 107 may be sanitation cloths 119, alcohol pads 121, and/or saturated wipes. Furthermore, kit 101 may include a sanitizing compound for dispensing onto the hands of the medical professional as a cleaning device. It is understood that devices 107 are not only useful for the tools and items used and the medical professional, but also for cleaning any necessary areas of the patients property to avoid contamination.

During assessments, sometimes items may necessitate the use of a bio-hazard bag 111 for disposal of biological substances that pose a threat to the health of living organisms. Kit 101 is configured to optionally include bag 111 for such purposes. Used items may be inserted into bag 111 immediately after use for disposal after the assessment is complete.

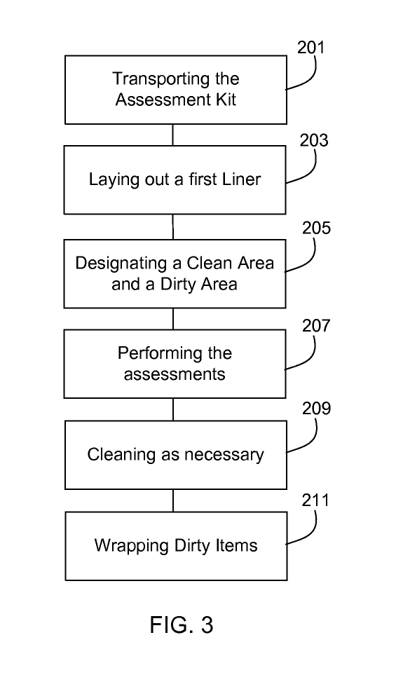

Referring now also to FIG. 3 in the drawings, a chart of the method of using the medical assessment kit of FIG. 1 is illustrated. Kit 101 may be used in different ways as alluded to above. Different assessments may require the use of a limited number of various tools and items. Therefore the exact method of using kit 101 may be varied. However, some of the basic steps are as follows. Kit 101 is transported into the home 201 of the patient where a first liner is laid out 203 to separate the kit from the property of the patient in the home. The kit is placed on the liner and a clean area is designated 205 for the placement of various tools and items prior to use. Additionally a dirty area is designated for the placement of used tools and items after use. The assessment is performed 207 and cleaning is done as necessary 209. Any dirty items not to be reused are wrapped 211 in the liner for disposal. Cross contamination is avoided by ensuring that the designated clean and dirty areas are properly utilized.

The dirty area is typically designated to be on a second liner that is required to be laid out for use. Note that use of the two designated areas allows for the transfer of tools and items between them as each are properly cleaned using the different cleaning devices. For example, a dirty tool may be cleaned with a sanitation cloth and then placed back within the bag 102 if it is not necessary for use later on. If later use is still required then it may reside in the dirty area awaiting the next assessment. Cleaning may be performed at the end of the assessment only or as the medical professional progresses through the assessment. Use of multiple gloves may be required to ensure that the cleaning devices and protection devices don't further contaminate areas outside the dirty area. Protection devices may be used by the medical professional during the assessment depending on the types of assessments being performed. Such devices may be interchanged at any time. Any use of a sanitizing compound (i.e. for hands) may be located on an external pocket of the bag 102 for easy access of the medical professional.

The current application has many advantages over the prior art including at least the following: (1) avoidance of cross contamination during the assessment; (2) an all-in-one portable kit for performing assessments that is in compliance with regulations; (3) ability to safely perform an assessment with decreased concern of contamination; and (4) reduced chances of being found in violation of the various regulations.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A medical assessment kit for ensuring compliance with regulations during an assessment of a patient, comprising:
a carrying device configured to hold items to provide a sanitary work space free of cross contamination;
a plurality of assessment tools configured to assess the patient;
a protection device to be worn by the medical professional to prevent contamination;
a cleaning device configured to sterilize the assessment tools after use; and
a liner configured to separate the assessment tools from contact with property of the patient.

2. The medical assessment kit of claim 1, wherein the carrying device is at least one of a bag, a case, and a box.

3. The medical assessment kit of claim 1, wherein the assessment tool is reusable.

4. The medical assessment kit of claim 1, wherein the protection device is a glove.

5. The medical assessment kit of claim 1, wherein the protection device is worn by the medical professional.

6. The medical assessment kit of claim 1, wherein the cleaning device is a sanitation cloth.

7. The medical assessment kit of claim 1, wherein the cleaning device is an alcohol pad.

8. The medical assessment kit of claim 1, wherein the cleaning device is a saturated wipe.

9. The medical assessment kit of claim 1, wherein the liner is configured to accept contaminated items for disposal.

10. The medical assessment kit of claim 1, further comprising:
a bio-hazard bag for disposal of biological substances that pose a threat to living organisms.

11. A method of performing a medical assessment of a patient in a home, comprising:
transporting an assessment kit into the home;
laying out a first liner configured to separate the kit from objects within the home;
designating a clean area and a dirty area, unused tools being located in the clean area when removed from the kit, used tools being located in the dirty area after use and prior to cleaning, the clean tools and the dirty tools being separated;
performing one or more assessments to the patient;
cleaning as necessary; and
wrapping dirty items in the first liner for disposal;
wherein cross contamination is avoided by maintaining a separate clean area and dirty area.

12. The method of claim 1, wherein the dirty area is located on a second liner.

13. The method of claim 11, further comprising:
using a protection device to protect a medical professional from contamination during the assessment of the patient.

14. The method of claim 11, wherein cleaning is performed by using at least a sanitation cloth and an alcohol pad.

15. The method of claim 11, wherein the step of cleaning is performed as a medical professional progresses through the stages of the assessment.

16. The method of claim 11, wherein the clean area and the dirty area are separated by the first liner from objects within the home.

17. The method of claim 11, further comprising:
interchanging the protection device worn by a medical professional during the assessment.

18. The method of claim 11, locating a sanitizing compound externally from the kit for access during the assessment.

19. The method of claim 11, wherein biological substances are disposed of in a bio-hazard bag.

20. The method of claim 11, wherein assessment tools are transferred between the clean area and the dirty area as used and cleaned.
